(19) United States
(12) Patent Application Publication
(10) Pub. No.: US 2006/0183216 A1
(43) Pub. Date: Aug. 17, 2006

(54) Containers for liquid storage and delivery with application to microfluidic devices

(76) Inventors: Kalyan Handique, Ann Arbor, MI (US); Aaron Kehrer, Ypsilanti, MI (US)

Correspondence Address: FISH & RICHARDSON P.C., PO BOX 1022, MINNEAPOLIS, MN, 55440-1022, US

(21) Appl. No.: 11/338,422

(22) Filed: Jan. 23, 2006

Related U.S. Application Data
(63) Continuation-in-part of application No. 11/281,247, filed on Nov. 16, 2005.
Application No. 11/281,247 is a continuation-in-part of application No. PCT/US05/15345, filed on May 3, 2005.
(60) Provisional application No. 60/645,784, filed on Jan. 21, 2005.

(51) Int. Cl. B67D 5/30; C12M 1/34

(52) U.S. Cl. 435/287.1; 222/14

(57) ABSTRACT

A container for a liquid reagent, wherein the container has an outer wall and an internal piercing member, such that, upon application of pressure to the outer wall of the container, the internal piercing member punctures the container from the inside, thereby liberating the liquid contained therein. Such a container is configured to store the liquid for periods between 6 to 18 months with minimal loss of the liquid inside, other than if the container is ruptured. Such a container is also configured to require a particular force to be applied to the outer wall to cause the internal piercing member to puncture the container, such a force being greater than that ordinarily experienced by the container during routine storage, transport, or handling. The container is preferably adapted for use with a microfluidic cartridge.

### PRIORITY

This application claims benefit of priority of U.S. provisional application Ser. No. 60/645,784, filed Jan. 21, 2005, incorporated herein by reference in its entirety. This application is also a continuation-in-part of U.S. nonprovisional application Ser. No. 11/281,247, filed Nov. 16, 2005, which itself is a continuation-in-part of international application serial no. PCT/US2005/015345, filed May 3, 2005, both of which are incorporated by reference in their entirety.

### FIELD OF THE INVENTION

The present invention generally relates to containers, such as blister-packages, for storing and dispensing a liquid reagent, and more particularly to application of such containers to microfluidic devices.

### BACKGROUND

A number of containers exist that permit storage, transportation, and delivery of liquids in all manner of volumes. In many circumstances it is important that the volume of liquid in question does not substantially diminish over time when the liquid is stored. In other circumstances, it is vital that some or all of the liquid is not lost from the containing device during transportation or handling. In still other circumstances, it is important that the volume of liquid in question can be dispensed from the containing device on demand, with little loss, and without introducing air bubbles.

Devices that rely on microfluidics for their operation would benefit from using reagent containers that offer at least the foregoing features. Nevertheless, certain requirements of the operation of microfluidic devices mean that containers having even these desired features are still not ideal for routine use.

Of considerable practical use today are systems that use microfluidic components to carry out real-time analysis of biological samples. See, e.g., U.S. patent application publication 2002/0143437 to Handique, et al., and U.S. Pat. No. 6,575,188 to Parunak, both of which are incorporated herein by reference in their entirety. An aspect of their convenience of use is the fact that they comprise a single bench-top unit that is configured to accept one or more disposable cartridges. The cartridges have microfluidic networks that permit reaction between volumes of reagents and the sample in a microfluidic network. The bench-top unit has the capacity to apply appropriate conditions such as temperature to the cartridge that results in the manipulations of the reagents and the sample necessary for analysis. The bench-top unit is also, usually, configured to make qualitative and/or quantitative measurements of the various reagents and the sample as reaction ensues. Since, ideally, an operator should only need to perform as few operations as possible, it would be desirable to limit such operations to introduction of the cartridge into the bench-top unit, and introduction of the sample into the cartridge, neither of which requires handling reagent solutions. In particular, it would be particularly advantageous if a liquid container for the other reagents could be developed that is integral with the cartridge and relieves the operator of the need to, in a separate step or steps, introduce each reagent into the cartridge.

The discussion of the background to the invention herein is included to explain the context of the invention. This is not to be taken as an admission that any of the material referred to was published, known, or part of the common general knowledge as at the priority date of any of the claims.

### SUMMARY

The present invention comprises at least the following items, as further described herein.

A container for a liquid reagent, wherein the container has an outer wall and an internal piercing member, such that, upon application of pressure to the outer wall of the container, the internal piercing member punctures the container from the inside, thereby liberating the liquid contained therein. Such a container is configured to store the liquid for periods of at least between 6 to 18 months with minimal loss of the liquid inside, other than if the container is ruptured. Such a container is also configured to require a particular force to be applied to the outer wall to cause the internal piercing member to puncture the container, such a force being greater than that ordinarily experienced by the container during routine storage, transport, or handling.

A container for dispensing a liquid reagent, comprising: a depressible dome having an outer surface, an inner surface, and an edge, wherein the inner surface comprises a protuberance having an apex; and a sealing membrane, affixed to the edge and encapsulating the liquid reagent between the membrane and the inner surface of the dome; wherein the protuberance is configured such that, upon application of a pressure to the outer surface of the dome, the apex of the protuberance pierces the sealing membrane, thereby dispensing the liquid reagent.

A microfluidic device, comprising: a substrate; a network of microfluidic channels disposed on the substrate, wherein the network has at least one opening that communicates with one of the microfluidic channels; and situated above the opening and in contact with the network, a container for dispensing a liquid reagent, wherein the container comprises: a depressible dome having an outer surface, an inner surface, and an edge, wherein the inner surface comprises a protuberance having an apex; and a sealing membrane having a first side and a second side, wherein the first side is affixed to the edge, and wherein the second side is affixed to the network, and wherein a liquid reagent is contained between the first side and the inner surface of the dome; and wherein the protuberance is configured such that, upon application of a pressure to the outer surface of the dome, the apex of the protuberance pierces the sealing membrane, thereby dispensing the liquid reagent into the opening.

A device for dispensing a liquid reagent, comprising: a depressible dome having an outer surface, an inner surface, and an edge; a sealing membrane, affixed to the edge and encapsulating the liquid reagent between the membrane and the inner surface of the dome; and a piercing element having an apex configured to pierce the sealing membrane, thereby dispensing the liquid reagent.

A reservoir for containing a liquid reagent until dispensed by piercing the reservoir with a piercing element, wherein the container has a wall such that, upon application of a piercing member to the wall of the container, the piercing member punctures the container, thereby liberating the liquid contained therein. Such a container is configured to store the liquid for periods of at least between 6 to 18 months with minimal loss of the liquid inside, other than if the container is ruptured. Such a container is also configured to require a particular force to be applied to the wall to cause the piercing member to puncture the container, such a force being greater than that ordinarily experienced by the container during routine storage, transport, or handling.

A method of dispensing a liquid reagent, comprising: applying pressure to an outer surface of a depressible dome, wherein the dome has an inner surface, and an edge, and wherein the inner surface comprises a protuberance having an apex; and wherein a sealing membrane, affixed to the edge encapsulates the liquid reagent between the membrane and the inner surface of the dome; wherein the protuberance is configured such that, upon applying the pressure to the outer surface of the dome, the apex of the protuberance pierces the sealing membrane, thereby dispensing the liquid reagent.

The details of one or more embodiments of the invention are set forth in the accompanying drawings and the description below. Other features, objects, and advantages of the invention will be apparent from the description and drawings, and from the claims.

Throughout the description and claims of the specification the word “comprise” and variations thereof, such as “comprising” and “comprises”, is not intended to exclude other additives, components, integers or steps.

### BRIEF DESCRIPTION OF THE DRAWINGS

FIG. 0A shows a reservoir having an internal piercing member.

FIG. 0B shows a microfluidic device having a reservoir and a hydrophobic vent.

FIG. 1 shows a microfluidic device in perspective view

FIG. 2 shows a microfluidic device in exploded view, including certain components thereof.

FIG. 3 shows an embodiment of a microfluidic device having a plurality of reservoirs.

FIG. 4 shows, in cross-sectional view, a microfluidic device having a reservoir with a piercing member.

FIG. 5 shows, in cross-sectional view, a microfluidic device having a reservoir with an integral piercing member.

FIG. 6 shows, in cross-sectional view, a microfluidic device having a reservoir with an integral piercing member that permits liquid to be loaded through it.

FIG. 7 shows, in cross-sectional view and plan view, a microfluidic device having a reservoir that is separated from a microfluidic network by a partition.

FIGS. 8A, 8B, and 8C show a reservoir having a plunger-like piercing member in, respectively, three different positions.

FIGS. 9A, 9B, and 9C show a reservoir having an integral plunger-like piercing member in, respectively, three different positions.

FIGS. 10A and 10B show a reservoir having an internal piercing member in, respectively, two different positions.

FIGS. 11A and 11B show a reservoir having a dome-like upper layer and an internal piercing member in, respectively, two different positions.

FIG. 12 shows a reservoir having a dome-like upper wall and a piercing member within the reservoir.

FIG. 13 shows a capsule-like reservoir configured to be ruptured by a piercing member.

FIGS. 14A and 14B show cross-sectional and plan views of a dome-like reservoir.

Like reference symbols in the various drawings indicate like elements.

### DETAILED DESCRIPTION

Microfluidic devices include devices with features having dimensions on the order of nanometers to 100's of microns that cooperate to perform various desired manipulations of liquids and gases. In particular, microfluidic devices perform material analysis and manipulation functions, such as chemical or physical analyses of samples, including biological samples. Microfluidic devices typically comprise one or more components that include, but are not limited to, channels, actuators such as gas actuators, vents, gates, valves, and chambers. Such components typically accommodate volumes of gas or liquid between a nanoliter and 100 microliters. The present invention provides reservoirs that can hold reagents in suitable volumes, and are suitably configured to introduce such reagents into a component of a microfluidic network. As referred to in the art, therefore, the reservoirs may be described as reagent inlets. As further used herein, the terms container and reservoir are used interchangeably.

Accordingly, the present invention relates to a reservoir capable of holding a liquid, e.g., a solvent, solution, buffer, a reagent, or combination thereof, and especially a reservoir adapted for use with a microfluidic device. The reservoir is configured to release the liquid upon application of a pressure and, optionally, use of a piercing member internal or external to the reservoir. The present invention also comprises microfluidic devices that include one or more such reservoirs each capable of holding a liquid, e.g., a solvent, solution, buffer, reagent, or combination thereof, configured to deliver such a liquid to a microfluidic component of the microfluidic device.

Exemplary liquids contained in the reservoir include water and aqueous solutions that include one or more salts, e.g., magnesium chloride, sodium chloride, sodium hydroxide, tris buffer, or a combination thereof. Such liquids are typically the reagents used in the art to dissolve lyophilized pellets that contain biological reagents such as enzymes, and PCR reagents. The liquids may also be those used to dissolve, dilute, or suspend biological samples.

A reservoir of the present invention includes a wall which can be manipulated, e.g., pressed, to decrease a volume within the reservoir, thereby causing a pressure to be applied to a liquid within the reservoir. In some embodiments, the application of pressure is sufficient to rupture some part of the reservoir, thereby releasing the liquid from within. In other embodiments, a piercing member, e.g., a needle-like member, ruptures a wall of the reservoir to release all or part of the liquid contained therein. The piercing member can be internal to the reservoir such that the piercing member ruptures the wall from an inner surface of the wall outwards. Alternatively, a piercing element may puncture the reservoir from outside it.

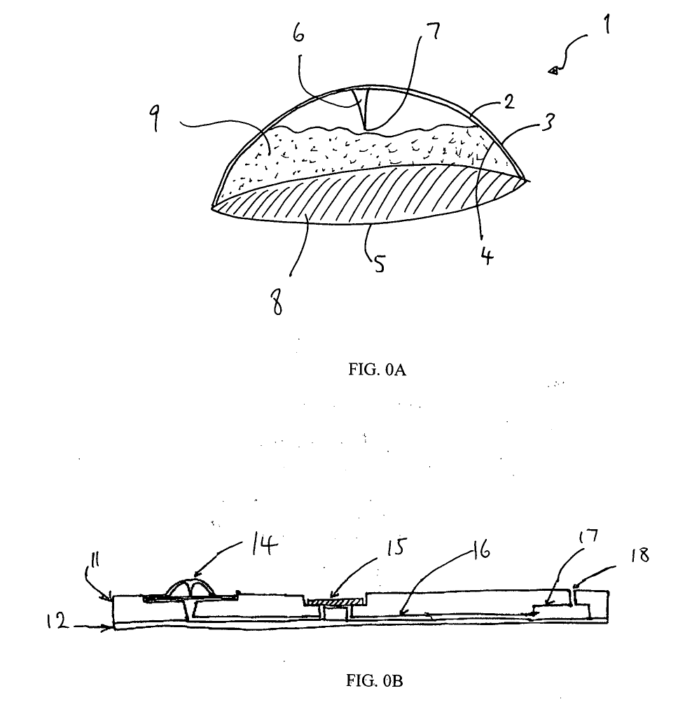

An exemplary embodiment of the present invention is shown in FIG. 0A, wherein: a reservoir 1 has a depressible dome 2. The dome 2 has an outer surface 3, an inner surface 4, and an edge 5, wherein the inner surface comprises a protuberance 6 having an apex 7; and a sealing membrane 8, affixed to the edge 5 and encapsulating the liquid reagent 9 between the membrane and the inner surface of the dome; wherein the protuberance 6 is configured such that, upon application of a pressure to the outer surface of the dome, the dome depresses such that the apex 7 of the protuberance pierces the sealing membrane, thereby releasing the liquid reagent.

As further described herein, the reservoir may be fabricated in several ways, including but not limited to: use of a single piece of material that is shaped to provide a closed volume that encloses the liquid within it; and use of two or more pieces of material that are bonded to one another to create a sealed volume that encloses the liquid within it.

In some embodiments, a maximum amount of liquid retained by a reservoir is less than about 1 ml. For example, a reservoir may hold 500 microliters or less, 300 microliters or less, 250 microliters or less, 150 microliters or less, or 100 microliters or less before a wall is depressed. Where such volumes are quoted as whole numbers, it is to be assumed that some variability, such as ±5% is within the scope of the invention: for example, a volume of 300 microliters encompasses volumes in the range 300±15 microliters. Generally, a reservoir holds at least 25 microliters, e.g., at least 50 microliters, or at least 100 microliters, where similar variability is permissible. The reservoir can introduce within about 10% of the intended amount of liquid, e.g., 50±5 μl. Typically, a reservoir is filled to within 75% of its actual volume. Thus, for example, a reservoir having a volume of 200 μl before actuation may receive 150 μl of liquid.

Upon depression of a wall, the volume of the reservoir is preferably deformed to around half its original volume.

It is consistent with the present invention that a microfluidic device, such as a microfluidic cartridge, can be equipped with two or more reservoirs as herein described, the reservoirs having different volumes from one another. Such a configuration facilitates storage and use of different reagents that are required in different quantities, but which can be delivered by employing the same mode of action. Accordingly, the embodiments described herein permit production of, e.g., a microfluidic cartridge, having two or more reservoirs that have different volumes from one another. Such embodiments represent advantages over devices in the art that are configured to accept, e.g., reagent-containing packs of a constant size. The reservoirs may also be of the same size, and hold the same volume as one another, in which case where the different receiving microchannels have varying volumes they may still receive the dispensed fluid but permit any excess to flow out to one or more waste chambers.

In some embodiments, the reservoir is dome-shaped. By this is meant that one wall of the reservoir is rounded in shape and, when placed in, e.g., a microfluidic cartridge, is raised above the level of the plane of the cartridge such that another wall is flat and is in contact with the cartridge. It is consistent with the reservoirs described herein that the term dome includes, but is not limited to, reservoirs having circular, approximately circular, elliptical, approximately elliptical, and cigar-shaped cross sections when viewed down an axis perpendicular to the plane of a microfluidic cartridge on which such a reservoir is situated. Such domes are also such that they are, when viewed along at least one axis parallel to the plane of a microfluidic cartridge on which they are situated, hemispherical, or approximately hemispherical, or have the shape of a cap of a sphere that occupies less than half of the volume of the sphere from which it is derived.

The reservoirs typically have a diameter of in the range of 0.3-1.5 cm, and may even be as wide as 2 cm. The reservoirs typically have a height of 0.2-0.6 cm, and may be as tall as 0.8 cm in certain embodiments. It would be understood that other variations of such dimensions are within the scope of the present invention, depending upon application.

In some embodiments, the reservoir of the present invention is similar to a “blister-pack” familiar to one of ordinary skill in the art of storing pharmaceutical products. In such instances, the reservoir has a dome, made from a depressible material and having an outer surface, an inner surface, and an edge. A sealing membrane, made from a second material, is affixed to the edge such that a liquid, such as a reagent, is encapsulated between the sealing membrane and the inner surface of the dome. Preferably the sealing membrane is made of aluminum foil sealed with a plastic coating. As further described herein, the inner surface of the dome comprises a protuberance having an apex configured such that, upon application of a pressure to the outer surface of the dome, the apex of the protuberance pierces the sealing membrane, thereby releasing the liquid reagent. It is especially preferable that the piercing element is integral to the material of the dome, and is centered thereon.

The size of the dome is important because the appropriate deformation pressure depends upon the area over which the force is applied. Certain formulae exist for predicting the deformation pressure required to depress a dome of given dimensions and thickness of material. See, e.g., Kaplan, A., Finite deflections and buckling of slightly curved beams and shallow shells under lateral loads, Ph.D. Thesis, Cal. Tech., Pasadena, Calif., 1954, incorporated herein by reference in its entirety. Presence of an internal piercing element, however, alters exact applicability of such formulae. In general, it is important that the force required for deformation be not so small that the reservoir cannot retain its shape and withstand various perturbations during storage and transit. However, the required force for deformation cannot be so great that an operator, including someone with weak hand muscles, cannot rupture the reservoir either by application of hand pressure, or by application of pressure from a mechanical device. Furthermore, it is important that the pressure required by, e.g., a mechanical device, is not so strong that structural damage occurs to, e.g., a microfluidic cartridge to which the reservoir is attached. Thus it is also important that the reservoir is not too small, so that forces applied to it result in pressures that are too high for it to withstand.

In another preferred embodiment, the force to be applied is 20-60 lbs/cartridge, or 5-12 lbs force/blister. The force will vary based on the thickness and properties of the sealing material, for a given dimension and material of the dome and apex.

The material from which at least one wall of a reservoir of the present invention is constructed is chosen because of certain desirable properties. In particular, the material has a flexibility that permits it to deform under application of a pressure from outside the reservoir. The reservoir therefore includes a wall that can be manipulated (e.g., pressed or depressed) to decrease a volume within the reservoir. In some embodiments, the wall lacks elasticity.

The wall may comprise, e.g., a metallic layer such as a foil layer, a polymer, or a laminate including a combination of layers of the same or different materials. Preferably the wall is made from a material selected from the group consisting of: polypropylene, PVC, PCTFE, and PVDC. Still other materials familiar to one of ordinary skill in the art are suitable. The preferred thickness of the wall depends upon the material from which it is composed.

In a preferred embodiment, the dome is made of polypropylene, by injection molding, and has a thickness of 200-300 μm.

Additionally, the wall is chosen from a material that resists passage of liquid or vapor therethrough. Preferably the materials of the various embodiments are chosen so that the device has a shelf-life of about a year. By this it is meant that the thickness of the various materials are such that the reservoir can retain the liquid without significant loss, e.g., through means such as diffusion or without substantial evaporation thereof, for a period of time, e.g., 6 months, or a year, or 18 months. In some embodiments, less than 10%, e.g., less than 5%, or less than 1% by weight, of the liquid evaporates or otherwise is lost from the reservoir over the period of time in question. Preferably the materials have a moisture vapor transmission rate of less than 1 g/m²/day, measured at 38° C. and 90% relative humidity (RH). Even more preferably the materials have a moisture vapor transmission rate of between 0.01 and 0.5 g/m²/day.

The reservoir also preserves the reactivity and composition of reagents contained therein. The reservoir is also preferably non-permeable to air, thereby minimizing the likelihood of oxidation of the liquid stored within. It is also important that the materials from which the reservoir is made are compatible with the particular reagent stored within. For example, a polymer that is susceptible to attack by alkaline solutions should not be used to store, e.g., NaOH solutions. Thus, the chemicals within the reservoir exhibit little or no change in reactivity over the period of time, e.g., 6 months, or a year, or 18 months, provided that the reservoir is not subject to undue extremes of temperature or pressure that cause the reservoir wall to degrade or to become more transmissive to liquid contained therein.

Actuation of the reservoir may include driving a piercing member through a wall of the reservoir. For example, the reservoir can include a piercing member (e.g., a needle-like or otherwise pointed or sharp member) that ruptures another portion of the reservoir (e.g., a portion of the wall) to release liquid. The piercing member can be internal to the reservoir such that the piercing member ruptures the wall from an inner surface of the reservoir (e.g., wall) outwards.

In some embodiments, a piercing member is located inside the reservoir. For example, the reservoir includes a first wall having an internal projection, which may be in contact with liquid in the reservoir. The reservoir also includes a second wall opposite the piercing member. The second wall can be either a separate piece of material or is a portion of the same piece of material from which the internal projection protrudes. During actuation of the reservoir, the piercing member is driven through the second wall, e.g., from the inside out, to release liquid. In some embodiments, a piercing member is an integral part of a first wall of the reservoir.

In some embodiments, the reservoir can be actuated to release liquid by pressing the wall, e.g., by an operator pressing his or her finger or thumb against an outer surface of the reservoir wall, or by mechanical pressure against the same. Mechanical pressure may come from, e.g., a flat member that is depressed by action of a lever upon a microfluidic cartridge having one or more reservoirs. The reservoirs, which are raised above the plane of the cartridge, are contacted by the flat member first. In this way, the pressure is applied directly to a wall of the reservoir. The pressure may also be applied to a plunger having a piercing member which punctures the reservoir wall from the exterior. In preferred embodiments, minimal pressure is required to actuate the reservoir. An automated system can be used to actuate, e.g., press upon, a plurality of reservoirs simultaneously or in sequence.

In some embodiments, the reservoir does not include a piercing member. Instead, internal pressure generated within the reservoir ruptures a wall of the reservoir allowing liquid to be released and to, e.g., enter the microfluidic device.

The reservoir can deliver a predetermined amount of liquid that is substantially gas-free, e.g., substantially air-free. Upon introduction of the liquid from the reservoir into a microfluidic component, the substantially air and/or gas free liquid produces few or no bubbles large enough to obstruct movement of the liquid within the microfluidic component. Use of a piercing member internal to the reservoir, as previously described herein, can enhance an ability of the reservoir to deliver substantially air and/or gas free liquids.

Upon actuating a reservoir to introduce liquid into the microfluidic device, liquid generally does not withdraw back into the reservoir. For example, upon actuation, the volume of the reservoir may decrease to some minimum but generally does not increase thereafter so as to withdraw liquid back into the reservoir. For example, the reservoir may stay collapsed upon actuation. In such embodiments, the flexible wall may be flexible but lack hysteresis or stretchiness. Alternatively or in combination, the reservoir may draw in air from a vent without withdrawing any of the liquid.

When using the reservoir in conjunction with a microfluidic device, it is particularly important that air trapped in the reservoir does not enter the microfluidic device along with the liquid from the reservoir, during activation thereof. Since it is difficult to fabricate a reservoir without automatically ending up with some residual air in the reservoir, a solution to this problem is to configure the microfluidic device so that a vent, such as a hydrophobic vent, permits such residual air to escape as soon as the reservoir is ruptured. Such an embodiment is shown in FIG. 0B, which shows a cross-section of a microfluidic device having a microfluidic substrate 11 on a laminate 12. A dome-shaped reservoir 14 with an internal piercing member is configured to release liquid into a microchannel 16. Air vent 15 has a hydrophobic membrane that permits gas but not the liquid to escape, thereby removing any air bubble coming from the reservoir upon depression. Air vent 15 is typically situated close by reservoir 14, at a distance that depends upon the configuration of other microfluidic circuitry on the chip. Such a distance may be of the order of 1 mm, and may be also be as long as 1 cm. A waste chamber 17 receives excess liquid and may also have an air vent 18 so that air in chamber 17 is expelled by displacement when the liquid enters.

When used in conjunction with a microfluidic cartridge, it is preferable that the reservoir is situated above a channel opening in the cartridge. Since it is also preferable that the reservoir deliver at least the right amount of liquid to the microfluidic network, the liquid is present in the reservoir in an amount substantially in excess of that required. Excess liquid that enters the microfluidic network is redirected to, e.g., a waste channel or outlet.

The reservoir can be assembled independently of a microfluidic device and then secured to the microfluidic device. In some embodiments, the wall is formed by vacuum formation, e.g., applying a vacuum and heat to a layer of material to draw the layer against a molding surface. The molding surface may be concave such that the wall is then provided with a generally convex surface.

In other embodiments, a reservoir having a dome and a sealing member may be constructed as follows: the dome-shaped member is inverted, filled approximately ¾-full with liquid, e.g., by using a syringe, and sealed by placing a sealing membrane on top, in contact with the edge of the dome. Sealing of the sealing membrane to the edge of the dome can take place by application of heat. The sealing membrane may then be attached, e.g., by gluing, to a microfluidic cartridge. The sealing membrane may comprise, e.g., a metallic layer such as a foil layer, a polymer, or a laminate.

A preferred embodiment of a blister pack configured to store liquids for extended periods on a microfluidic cartridge consists of a plastic thermoformed well made from a low moisture vapor transmission rate (MVTR) material, e.g., Aclar, metallized (e.g., Al) laminate, a plastic or metal piercing barb, and a foil laminate with a low MVTR that is easily piercable. The sealing membrane may also be made of Al foil coated with a plastic film that is pierceable and meets MVTR requirements further described herein. The pouch assembly is designed to store up to 200 microliters of fluid with the piercer accounting for 50 microliters of that volume.

The reservoir is filled, sealed, and attached to the microfluidic interface. The design is such that when the optical/pressure jig is placed on the cartridge the pouches will collapse, pushing a piercing member through the foil and forcing the stored liquids into the device.

### EXAMPLES

### Example 1

### Microfluidic Device

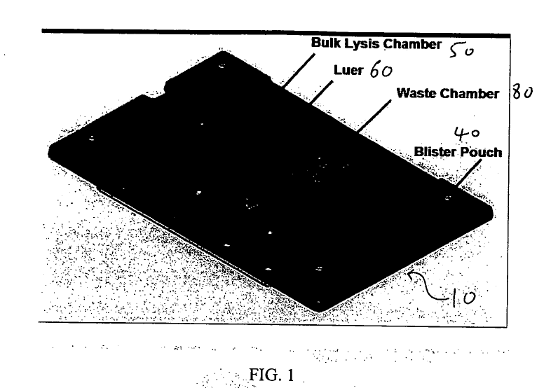

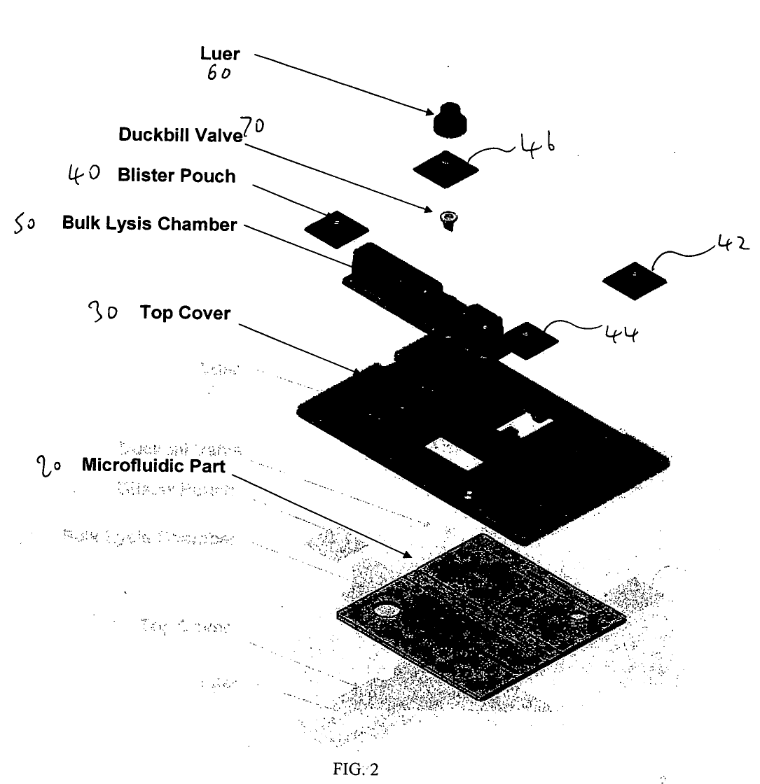

Referring to FIGS. 1 and 2, a microfluidic device 10 includes a microfluidic part 20, e.g., a substrate, at least partially defining a microfluidic network, and a cover 30, which may seal portions of the microfluidic network. A reservoir, e.g., a blister pack 40, 42, 44, or 46, holds liquids until released by a user, e.g., by automatically or manually applying pressure to the blister pack, either in turn or to more than one simultaneously. Upon applying pressure, the blister pack releases a predetermined amount of liquid into, e.g., a channel or chamber of the microfluidic device. The predetermined liquid can combine with, e.g., a dry reagent or sample, to prepare, e.g., a mixture having a known volume and/or concentration.

The microfluidic device also typically includes a bulk lysis chamber 50 for releasing contents of cells and other biological particles. A luer fitting 60 and valve 70 allows one-way introduction or removal of material to and from the lysing chamber. A waste chamber 80 collects, e.g., excess reagent solutions. Microfluidic devices including lysing chambers are described in international application number PCT/US2004/025181, which application is incorporated herein by reference.

### Example 2

### Microfluidic Device Having a Plurality of Reservoirs

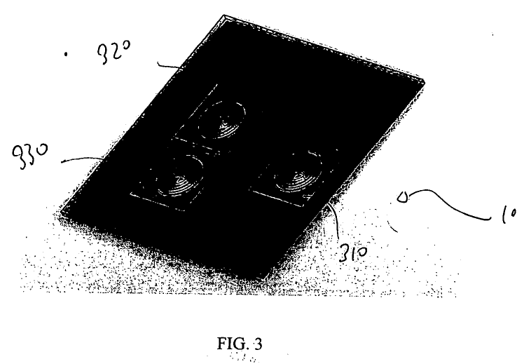

Referring to FIG. 3, an embodiment of a microfluidic device 10 having a plurality of reservoirs 310, 320, and 330, is shown. In some embodiments, the reservoirs, e.g., blister packs, provide about 50 microliters of fluid after any withdrawal. For dome materials that are not irreversibly deformed, such as due to elasticity of the laminate from which they are made, the blister will retain its original shape after removing the applied force. This may cause some suck-back of liquid unless the air pressure is properly vented from the device.

### Example 3

### Reservoir Having a Dome with a Piercing Member

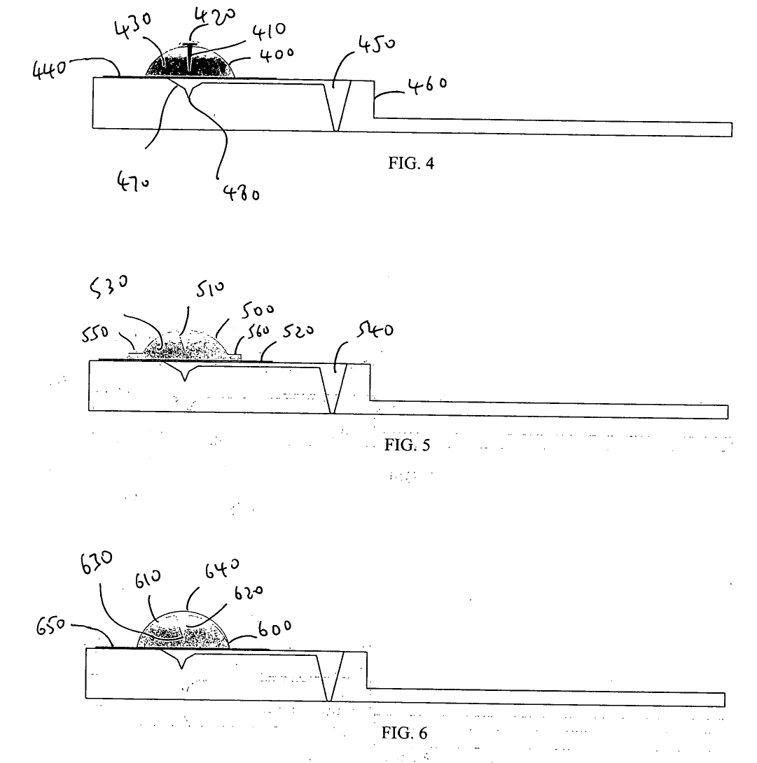

Referring to FIG. 4, a reservoir has a dome 400 that includes a piercing member, e.g., a pin 410, extending into the reservoir and sealed with, e.g., epoxy 420. The reservoir retains liquid 430. A layer, e.g., a foil laminate 440, underlies the reservoir. When the piercing member 420 is depressed, the dome deforms and the piercing member ruptures the layer 440 from an inner surface of the layer outward allowing liquid to enter the microfluidic network 450.

The substrate 460 can be configured so that the hole 470 directly underneath pin 410 contains a recess 480 that accommodates the pin when it is depressed. Such a recess, although not explicitly referenced in every instance, can also be found in other embodiments of the invention, as further described herein.

### Example 4

### Reservoir Having a Dome with an Integral Piercing Member

Referring to FIG. 5, a reservoir 500 has a dome that includes a piercing member integral with the dome. For example, the piercing member 510 can be formed by a pointed internal wall of the reservoir. The piercing member can rupture a layer, e.g., a laminate 520 located opposite the piercing member and separating the liquid 530 within the reservoir from the microfluidic network 540. The reservoir can be formed, sealed to a foil laminate, filled through a hole in one side. In this way, liquid can be loaded through a hole 550 on one side of the dome and gas can be simultaneously vented through a hole 560 on the other side. Afterwards, the holes are capped or plugged. This loading mechanism is especially useful when the sealing membrane is attached to the dome before loading of liquid into the blister.

### Example 5

### Reservoir with a Dome Having an Integral Piercing Member with a Hole

Referring to FIG. 6, a reservoir 600 includes a dome 610 in which the piercing member 620 is integral with the material of the dome. The piercing member has a concentric hole 630 through which the liquid can be loaded. The dome is capped with another material 640, such as a laminate, that seals the hole through which the liquid was loaded. The reservoir is sealed to a foil laminate 650.

### Example 6

### Reservoir Having a Partition

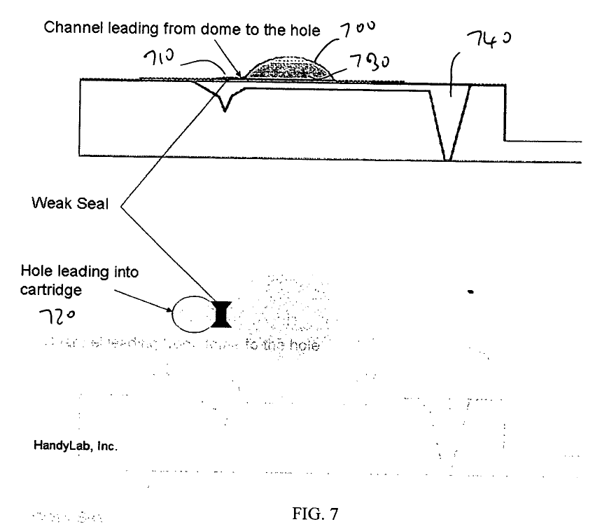

Referring to FIG. 7 a reservoir 700, without an internal piercing member, includes a partition 710 that obstructs a hole 720 that separates liquid 730 in the reservoir from the microfluidic network 740. The partition can rupture without being pierced by a piercing member. Partition 710 acts as a weak seal that breaks when pressure is applied to the reservoir 700.

### Example7

### Reservoir and Plunger

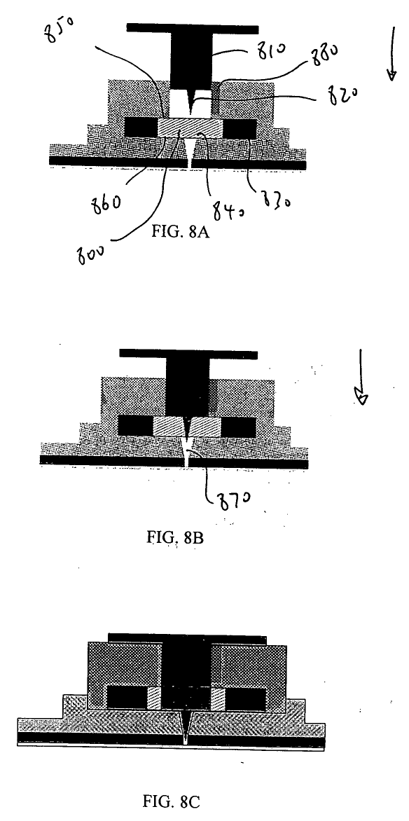

Referring to FIGS. 8A, 8B, and 8C, a reservoir 800 operates in conjunction with a plunger-like member 810 having a piercing element 820. Reservoir 800 has upper and lower layers, e.g., upper and lower laminate or foil layers, which act as moisture vapor barriers. Liquid 840 is disposed between the upper 850 and lower 860 layers. The reservoir is optionally surrounded by a supporting structure, e.g., a toroidal gasket 830, which supports the upper and lower layers at its upper and lower opposed surfaces. In FIG. 8A, the plunger has not been depressed. Vertical arrows in FIGS. 8A and 8B denote the direction of motion of plunger 810 for the purpose of piercing reservoir 800.

In FIG. 8B, the piercing member 810 has been depressed until the piercing member has pierced both the upper and lower layers of the reservoir, thereby bringing the liquid into communication with the microfluidic network 870. A vent 880 adjacent the plunger allows gas trapped between the piercing member and the upper layer of the reservoir to escape without being forced into the microfluidic network.

In FIG. 8C, the piercing member has been fully actuated. A portion of the piercing member displaces a predetermined volume of liquid from the reservoir and introduces the predetermined volume of liquid into the microfluidic device. It is preferable to displace the plunger to this extent so that pressure can force liquid from the reservoir into the microfluidic network.

### Example 8

### Reservoir with Integral Plunger

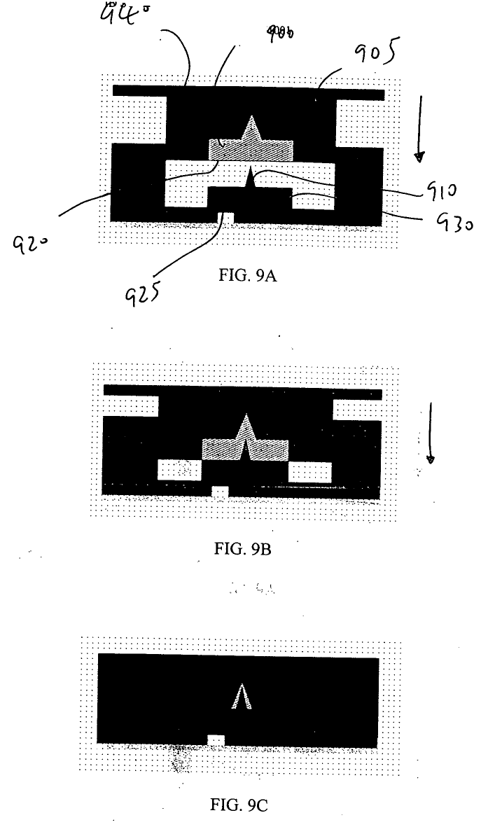

Referring to FIGS. 9A, 9B, and 9C, a reservoir 900 is integral with a movable plunger 905. A fixed piercing member 910 is located to rupture a lower layer 920 that confines the liquid in the reservoir. A passage 925 having an opening adjacent the piercing member is in fluidic communication with the microfluidic network to permit liquid form the reservoir to dispense into the network. The piercing member is supported by a piercing member support 930 having a shape generally complementary to an interior of the reservoir. Vertical arrows in FIGS. 9A and 9B denote the direction of motion of plunger 905 for the purpose of piercing reservoir 900.

In FIG. 9B, the plunger has been depressed so that the piercing member has just ruptured the lower layer of the reservoir. Plunger 900 has a vent 940 that permits air trapped between the plunger and the piercing element to be vented as the plunger is depressed.

In FIG. 9C, the reservoir has been fully depressed onto the piercing member and piercing member support. The volume of fluid displaced from the reservoir is generally determined by the size of the piercing member support.

### Example 9

### Reservoir with External Actuator

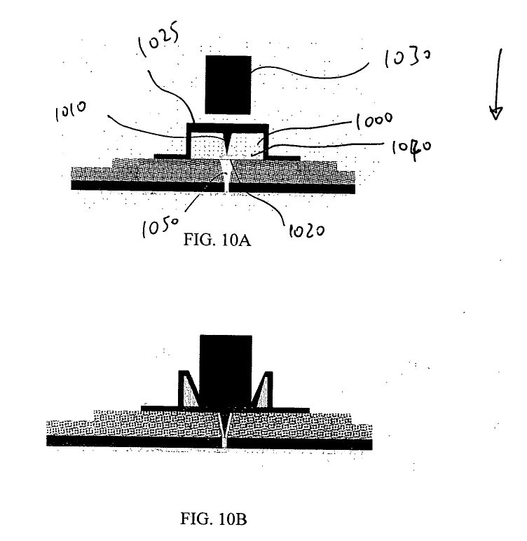

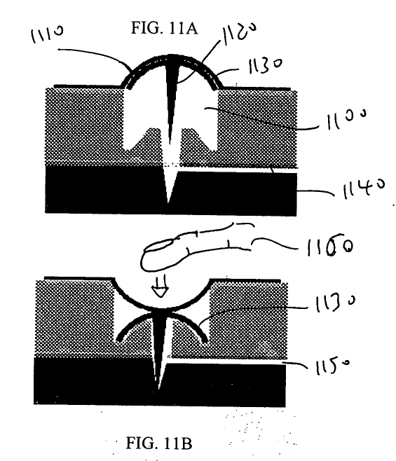

Referring to FIGS. 10A and 10B, a reservoir 1000 holding liquid 1040 includes an internal piercing member 1010 and a lower wall defined by a layer 1020, e.g., a laminate or foil that acts as a moisture vapor transmission barrier. The rest of the reservoir is defined by a depressable material 1025, such as has been further described herein. The vertical arrow in FIG. 10A denotes the direction of motion of plunger 1030 for the purpose of piercing reservoir 900.

In FIG. 10B, an actuation member 1030 has been driven into the reservoir, driving the piercing member through the lower layer and bringing liquid within the piercing member into contact with the microfluidic network 1050.

### Example 10

### Reservoir with Internal Piercing Member

Referring to FIGS. 11A and 11B, a reservoir 1100 includes a dome-like upper layer 1110, and an inner layer 1130 with an internal piercing member 1120. The dome-like upper layer inverts upon actuation, e.g., by finger 1160, as in FIG. 11B, and forces the separate inner layer 1130, to which the internal piercing member is attached, downwards through a sealing layer 1140. Liquid in the reservoir is thereby released into a channel 1150 of a microfluidic network. Although, in FIGS. 11A and 11B, piercing member is shown as integral to inner layer 1130, it will be readily appreciated that the two may be made of separate parts, joined to one another.

### Example 11

### Reservoir and Guide Channel for Piercing Member

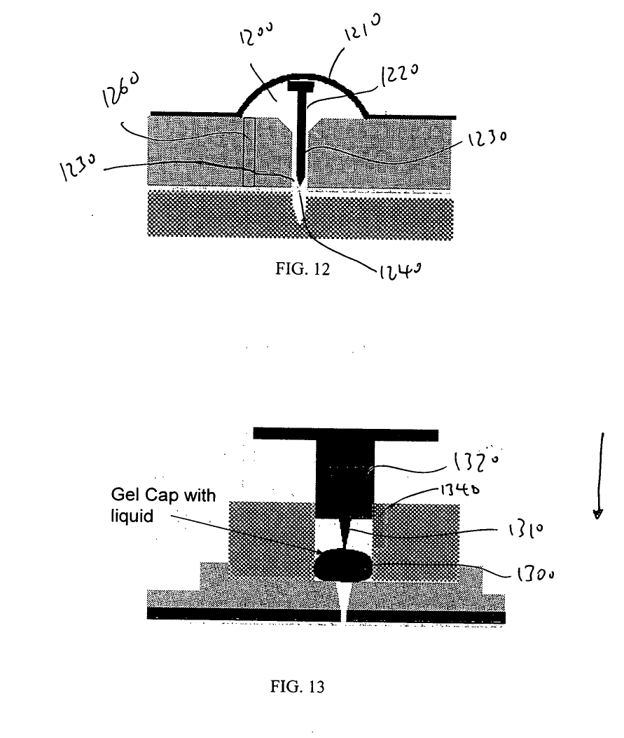

Referring to FIG. 12, a reservoir 1200 is defined by a dome-like upper wall 1210. A piercing member 1220 is present within the reservoir, in contact with, or attached to, the upper wall. A channel 1230 of the microfluidic device maintains the position of the piercing member but allows the piercing member to slide when depressed to rupture a sealing layer 1240 that separates the reservoir and the microfluidic device 1250. A loading channel 1260 allows liquid to be introduced to the reservoir.

### Example 12

### Reservoir in a Capsule with External Piercing Member

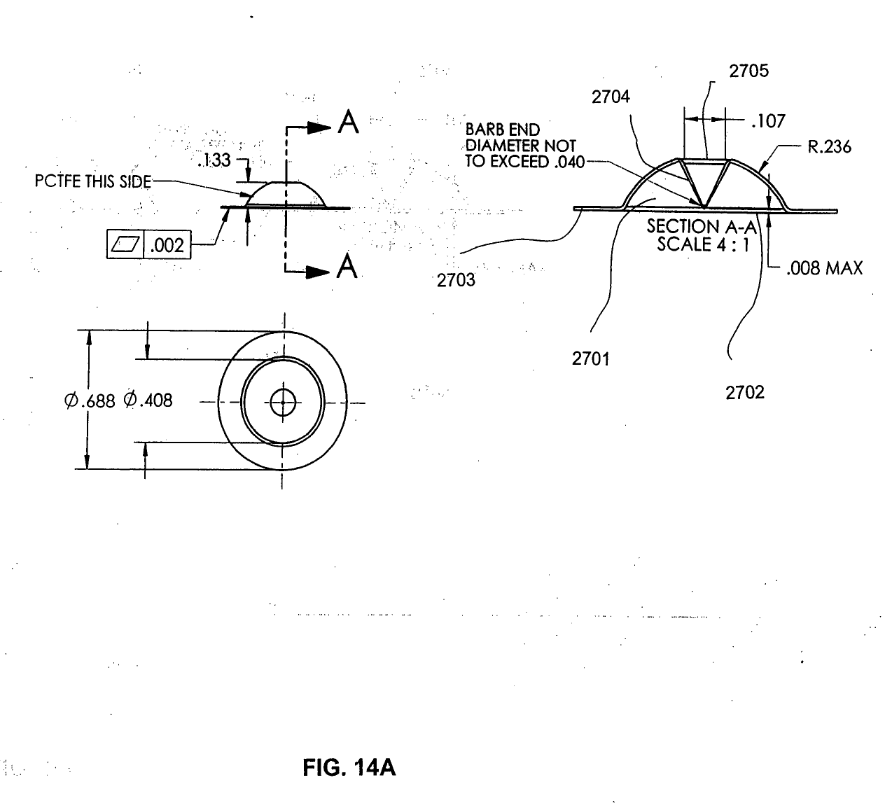

Referring to FIG. 13, a reservoir is defined by a capsule 1300, e.g., a gel capsule, which is ruptured by a piercing member 1310 attached to an external plunger 1320. A vent 1340 permits air to escape while plunger 1320 is depressed, in the direction of the vertical arrow shown. The gel cap is made of a material having a thickness such that it has an effective shelf-life of 6-18 months. The gel cap is rounded in shape and preferably made from a single piece of material.

### Example 13

### Microfluidic Network

Microfluidic networks suitable for use with the reservoirs of the present invention can be found in U.S. application Ser. No. 11/281,247, filed Nov. 16, 2005, to which the instant application claims priority.

### Example 14

### Reservoirs with Integral Piercing Member

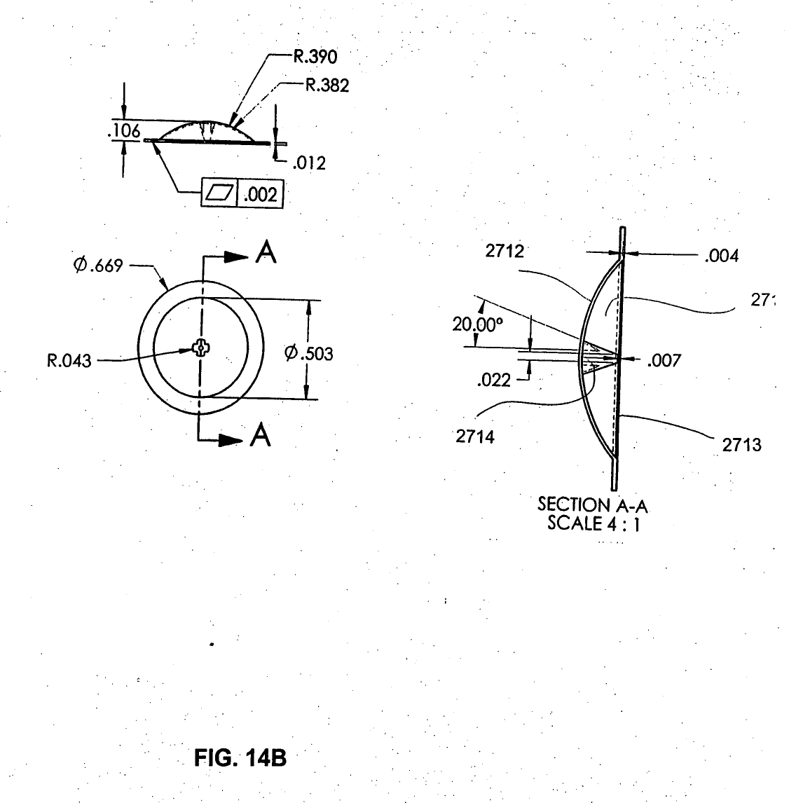

A still further embodiment of a reservoir with a piercing member is shown in FIG. 14A which shows a reservoir 2701 having an outer shell 2703 and a piercing element 2704 that are both made of the same piece of material. Such a combined shell and piercing element can be formed from many processes known to one of ordinary skill in the art. Especially preferred processes are vacuum thermo-forming and injection moulding. Piercing element 2704 is generally conical in shape, with the apex adjacent to a membrane 2702; its apex preferably does not exceed 0040″. The piercing element will puncture membrane 2702 and release liquid from reservoir 2701 when the outer shell is depressed. Representative dimensions are shown on FIG. 14A. The reservoir may be constructed so that the upper surface is level, with a flat protective piece 2705 covering the base of the conical shape of piercing element 2704. The embodiment shown in FIG. 14A is formed by thermoforming in which the piercing element is not a solid item but is part of a film that has been manipulated into the shape shown.

Yet another embodiment of a reservoir with a piercing member is shown in FIG. 14B showing a reservoir 2711 having a single-piece outer shell 2712 and piercing element 2714. Such a combined shell and piercing element can be formed from many processes known to one of ordinary skill in the art. Especially preferred processes are vacuum thermo-forming and injection moulding. Piercing element 2714 can be frustoconical in shape, with its narrower side adjacent to membrane 2713. Alternatively, piercing element 2714 can comprise several separate piercing elements, arranged within a conical space. Preferably there are four such piercing elements where multiple elements are present. The embodiment shown in FIG. 14B is formed by injection molding in which the piercing element is filled with plastic.

It is to be understood that the dimensions of the reservoir, piercing element, shell and moulding shown in FIGS. 15A and 15B as decimal quantities in inches are exemplary. In particular, the dimensions are such that the shell does not collapse under its own weight and is not so as strong to prohibit depression of the piercing member when required during operation of the device.

A number of embodiments of the invention have been described. Nevertheless, it will be understood that various modifications may be made without departing from the spirit and scope of the invention. Accordingly, other embodiments are within the scope of the following claims.

What is claimed is:

1. A container for dispensing a liquid reagent, comprising:
a depressible dome having an outer surface, an inner surface, and an edge, wherein the inner surface comprises a protuberance having an apex; and
a sealing membrane, affixed to the edge and encapsulating the liquid reagent between the membrane and the inner surface of the dome;
wherein the protuberance is configured such that, upon application of a pressure to the outer surface of the dome, the apex of the protuberance pierces the sealing membrane, thereby dispensing the liquid reagent.

2. The container of claim 1, wherein the liquid reagent has a volume of between 100 and 200 μl.

3. The container of claim 1, wherein the container has a volume of between 200 and 300 μl.

4. The container of claim 1, wherein the depressible dome is made of a material selected from the group consisting of: polypropylene, PVC, PCTFE, and PVDC.

5. The container of claim 1, wherein the depressible dome is made of a material having a thickness of 200-300 μm.

6. The container of claim 1, wherein the depressible dome is made of a material having a moisture/vapor transmission rate of less than 1 g/m²/day.

7. The container of claim 1, wherein the pressure is 5-12 lbs force.

8. The container of claim 1, wherein the sealing membrane is comprised of aluminum foil with a plastic coating.

9. The container of claim 1, wherein the protuberance is situated in the center of the dome.

10. The container of claim 1 wherein the dome is approximately circular, elliptical, or cigar-shaped.

11. The container of claim 1 wherein the protuberance is approximately conical.

12. The container of claim 1 wherein the apex has a diameter of 200 micrometer.

13. The container of claim 1 wherein the protuberance is approximately 1 mm in height above the inner surface.

14. A microfluidic device, comprising:
a substrate;
a network of microfluidic channels disposed on the substrate, wherein the network has at least one opening that communicates with one of the microfluidic channels;
and
situated above the opening and in contact with the network, a container for dispensing a liquid reagent, wherein the container comprises:
a depressible dome having an outer surface, an inner surface, and an edge, wherein the inner surface comprises a protuberance having an apex; and
a sealing membrane having a first side and a second side, wherein the first side is affixed to the edge, and wherein the second side is affixed to the network, and wherein a liquid reagent is contained between the first side and the inner surface of the dome;
and wherein the protuberance is configured such that, upon application of a pressure to the outer surface of the dome, the apex of the protuberance pierces the sealing membrane, thereby dispensing the liquid reagent into the opening.

15. The microfluidic device of claim 14, wherein the opening is configured to cause air from the container to vent from the device through a hydrophobic valve.

16. The microfluidic device of claim 14, wherein the one of the microfluidic channels communicates with a waste chamber so that excess liquid reagent is directed to the waste chamber.
